(19) United States
(12) Patent Application Publication
(10) Pub. No.: US 2013/0201447 A1
(43) Pub. Date: Aug. 8, 2013

(54) REFRACTOMETER WITH A COMPARATIVE VISION CORRECTION SIMULATOR

(71) Applicant: DigitalVision, LLC, Atlanta, GA (US)

(72) Inventors: Keith P. Thompson, Atlanta, GA (US); Jose R. Garcia, Mableton, GA (US)

(73) Assignee: DigitalVision, LLC, Atlanta, GA (US)

(21) Appl. No.: 13/758,597

(22) Filed: Feb. 4, 2013

Related U.S. Application Data
(60) Provisional application No. 61/594,784, filed on Feb. 3, 2012.
(60) Provisional application No. 61/594,772, filed on Feb. 3, 2012.

(51) Int. Cl. A61B 3/02

(52) U.S. Cl. CPC A61B 3/02
USPC 351/201; 351/237; 351/246

(57) ABSTRACT

A method and apparatus for vision testing and for simulation of eyesight correcting modalities is disclosed, the method including generating one or more images to be viewed by a patient, modulating the wavefront of each image by a differing amount and/or changing other optical attributes of one or more images by differing amounts, and selecting the preferred image based upon patient response. The apparatus includes devices for generating one or more images to be viewed by a patient, modulating the wavefront of each image by a differing amount and/or changing other optical attributes of one or more images by differing amounts, and devices for selecting the preferred image based upon patient response.

### FIELD OF INVENTION

This invention relates to subjective, monocular, or binocular, patient-interactive vision testing and comparative simulation of vision provided by eyesight-correcting modalities with different specifications.

### DESCRIPTION OF THE PRIOR ART

The phoropter lens dial such as the one described in U.S. Pat. No. 4,523,822 is the most common vision testing device in present use. The phoropter is comprised of dials of lenses of fixed spherical and cylindrical power that vary in 0.25 D or 0.125 D increments. During vision testing, the phoropter is placed in front of the patient's eyes and different lenses are dialed into the device's viewing aperture while the patient views letters on an eye chart through the selected lenses. Based upon an increase or decrease in the patient's perceived clarity of the letters with each combination of lenses, the refractionist iteratively determines the best combination of spherical and cylindrical lenses to correct eyesight and records these values as the optical specifications for eyeglasses that are prescribed for the patient. This information is also used to specify the optical properties for contact lenses and for the laser ablation profiles in some laser vision surgery treatments such as PRK and LASIK. In the case of laser vision surgery, the laser treatment changes the curvature of the anterior corneal surface which reduces or eliminates the focusing error of the eye. Those skilled in the art write prescriptions for conventional eyeglasses, contact lenses, and laser vision surgery in units of dioptric power, “D” in increments of 0.25 D or 0.125 D resolution (a lens with +1 Diopter of optical power focuses parallel light at 1 meter).

Practicing clinicians skilled in the art know that the method of vision testing using the phoropter has deficiencies that include, among others, a measurement resolution that is limited by the differences in power of its fixed power spherical and cylindrical lenses (typically 0.125 or 0.25 D), the inability to measure higher order aberrations such as spherical aberration, coma, trefoil, and other aberrations; the requirement for the patient to remember what the preceding image looked like when comparing it to the present image, and the placement of a bulky optical device in immediate proximity to the patient which can induce instrument accommodation errors.

The process of peering at black and letters on a white eye chart through the phoropter's small apertures while maintaining a fixed head position is an unnatural condition that fails to replicate the patient's day-to-day visual tasks. Moreover, the optical attributes of corrective modalities other than their refractive properties, such as photochromic, anti-reflective, and other premium lens coatings cannot be demonstrated using the phoropter and similar prior art methods. Therefore, the testing of vision and the specification of eyesight correcting modalities using the conventional phoropter and eye chart has well known deficiencies and limitations.

In U.S. Pat. No. 5,777,719, Williams disclosed a wavefront sensor for determining the wave aberrations of the living eye by using the Hartmann-Shack method of analyzing light from a reflected point source image of the retina. Since Williams's disclosure, numerous US Patents have been granted for methods and apparatuses for measuring vision and devising corrective modalities based upon objective aberrometry that do not incorporate interactive patient feedback.

In U.S. Pat. Nos. 7,703,919B2 and 7,926,944B, the inventors disclose disadvantages of using vision measurements to specify eyesight-correcting modalities based upon objective aberrometers, such as Hartmann-Schack devices, and teach a new visual metric based upon the neuro-ocular wavefront as defined in the '919 and '944 patents.

Regardless of the vision metric that is used to create an optical specification for a corrective modality, doctors and patients may find it desirable to demonstrate, or to simulate, the image forming properties of this specification to the patient before the modality is prescribed. To perform this simulation it is necessary to modulate the wavefront of an image to the same degree as it will be modulated by a corrective modality with a particular optical specification and then project the image on the patient's retina and obtain subjective feedback from the patient regarding the quality of the image. Several prior art disclosures teach such methods of simulating a corrective modality.

In U.S. Pat. Nos. 6,722,767 and 6,997,555 assigned to Zeiss/Meditec, an apparatus and method is disclosed for generating a single image for viewing by an eye of a patient, modulating the wavefront of that image by an adaptive optic or similar means, and projecting the image onto the retina for the patient to appraise the degree to which the image is distorted or clear. The Zeiss disclosures teach that the degree of distortion of images produced by different wavefront modulations is compared by the patient in a sequential fashion. By subjectively appraising and comparing the distortions of these images caused by difference modulations of the wavefront of the images, a wavefront modulation that provides the sharpest image is arrived at sequentially in an iterative fashion. The method taught in the Zeiss disclosure is similar to the iterative method of subjective refraction with a phoropter described above except that the Zeiss disclosures teach a means to modulate the wavefront of the image to include higher order aberrations with an adaptive optic system, whereas the phoropter is limited to imparting modulations to the wavefront of the image that are limited to spherical and cylindrical changes. The final wavefront modulation selected can be used as a basis for the specification of an eyesight-correcting modality, according to the Zeiss disclosure.

One disadvantage of the Zeiss disclosures is that a method and apparatus is taught for projecting an image under monocular viewing conditions only. It is known to those skilled in the art that normal human vision is binocular in nature and that the viewing of an image by one eye may influence both the focusing properties of the patient's fellow eye and the image that is perceived by a conscious patient through the actions of the higher order visual pathways in the retina and brain.

In U.S. Pat. No. 6,827,442 B2 assigned to Johnson & Johnson, a method of presenting an image with a modulated wavefront on the patient's retina for subjective assessment is described in which the image modulation is based upon ophthalmic wavefront measurements made by a Shack-Hartmann, or similar, objective aberrometery device. The objectively measured aberration is then used to modulate the wavefront of the image which is projected onto the retina by a suitable adaptive optic device similar to that described by Zeiss. The Johnson & Johnson disclosure taught the use of a binocular method of projecting a modulated wavefront of an image onto the retina, thereby overcoming the monocular limitation inherent in the Zeiss disclosures.

Artal, in European Patent No. EP2471440A1, disclosed a phoropter device for subjective vision testing that incorporated digital phase control technology. As explained in Artal's disclosure:
Thus, it is an electro-optical phoropter with a technology based on digital phase control. Therefore the invention also refers to a method that incorporates what may be identified as wavefront engineering. The present invention likewise enables the simulation of vision by means of any optical element. Thus it is related to the so called visual simulators. In particular, the instrument has the possibility of generating scenes that are perceived by the patient in a three dimensional manner during the measurement of the refraction or the simulation of ophthalmic elements, all of the foregoing in an electro-optical manner. The invention is related to the subjective measurement of the visual quality of the subjects and the limits of their vision, all in a binocular manner.

Artal's disclosure taught a means to simulate vision provided by a corrective modality that included the ability to modify the wavefront of the image with higher order aberrations that prior art phoropters could not impart. Unlike the Johnson & Johnson disclosure, Artal's device did not require the use of an objective aberrometer to acquire a measurement of the ophthalmic wavefront. Rather, it employed a phase modulator that modulated the wavefront of an image that was directed to the retina followed by a subjective assessment by the patient concerning the quality of the image. Unlike the Zeiss disclosures, Artal's device provided for binocular testing.

In the phoropter, and in similar prior art methods, and in the disclosures by Zeiss, Johnson & Johnson, and Artal, the corrective lenses of the devices are required to be placed in immediate proximity to the patient's eyes. It is well known to those skilled in the art that such proximate location has significant disadvantages that include, among others, the propensity to cause instrument accommodation errors, reduction of the patient's field of view, and the inability to obtain vision measurements or to simulate an eyesight-correcting modality of a particular specification under natural viewing conditions.

In U.S. Pat. No. 3,874,774, Humphrey described a subjective, binocular vision testing instrument known as the Humphrey Vision Analyzer (“HVA”) in which the corrective lenses were located remotely in a cabinet that was interposed between the patient and the operator. Alvarez adjustable spherical and cylindrical lenses were used in the device, and they were imaged—or optically relayed—to the appropriate plane near the patient's eye by a concave field mirror that was located approximately 3 Meters in front of the patient. Humphrey referred to this arrangement as a “phantom lens architecture” and it eliminated the need to place a bulky apparatus holding the corrective lenses in proximity to the patient. When viewing images in the HVA field mirror, it appeared to the patient as if invisible “phantom” corrective lenses were placed before his eyes and it permitted vision testing to be conducted under natural viewing conditions without the inducement of instrument accommodation, a common source of error inherent with prior art testing devices including the phoropter, the Zeiss, Johnson & Johnson, and Artal disclosures cited above.

Although the Humphrey disclosure resolved the disadvantages of placing corrective lenses in immediate proximity to the patient that was inherent in prior art methods, the HVA's dioptric resolution was no better than that of a phoropter because the device's adjustable lenses were used to emulate an ophthalmological prescription with a maximum measurement resolution of 0.125 D. The HVA lacked optical components necessary to obtain refractive metrics other than sphere and cylinder such as higher order aberrations or the neuro-ocular wavefront error. The HVA employed a field mirror that induced aberrations and astigmatism that were difficult to correct, it required a complicated method of setting astigmatic power, and it interposed a bulky desk between the patient and the doctor that precluded the doctor's access to the patient and the use of his examination instruments.

To overcome these and other limitations with the U.S. Pat. No. 3,874,774 device, a novel method and apparatus for vision testing was disclosed in the Applicant's co-pending U.S. patent application Ser. No. 13/738,644 entitled A REFRACTOMETER WITH A REMOTE WAVEFRONT GENERATOR which is included as a reference as if it were appended herein in its entirety. The '13/738,644 application discloses a wavefront generator capable of modulating the wavefront of image to spherical and cylindrical resolutions greater than that of prior art (generally limited to 0.125 or 0.25 D) and that is also capable of modulating the wavefront of an image to encompass higher order aberrations such as spherical aberrations, coma, and others. The '644 disclosure also taught means to remotely relay the wavefront generator to a plane on or near the patient's eyes without the undesirable induction of astigmatism and higher order aberrations inherent in the Humphrey method. It further taught the use of an eye tracker to improve measurement accuracy and to permit normal patient head and eye movement during the exam, free from the need of restraining devices required by prior art devices. The '644 disclosure also taught a novel configuration to the device with a much smaller instrument footprint and the ability for the doctor to interact directly with the patient and use his examination instruments, features the '744 device lacked.

While the '644 disclosure was a substantial improvement over the prior art '744 disclosure and other prior art vision testing methods, it was discovered during patient testing that the patient's ability to detect small differences in sphero-cylindrical and/or higher order wavefront modulation was enhanced by projecting two or more images that had different modulations to their wavefront on a substantially simultaneous basis for concurrent comparison by the patient. Thus, it was discovered that the '644 disclosure could be improved if it were modified to permit patients to compare images on a substantially simultaneous and optionally side-by-side basis. Such a simultaneous comparative capability is lacking with the conventional phoropter, and, with the prior art vision simulation methods of Zeiss, Johnson & Johnson, and Artal, discussed above. Patients undergoing a vision examination with any of these prior art methods must “remember” what the preceding image looked like in order to compare it to the current image. Patients often find subtle differences in image quality to be very difficult to discern when they are viewed sequentially rather than simultaneously, and thus the results of vision measurements and simulations of corrective modalities using these prior art methods have inherent limitations.

In U.S. Pat. No. 3,240,548 to Biessels, an optical device was disclosed that allowed patients to compare two identical images formed by a single object after each image passed through corrective lenses of different spherical or cylindrical powers. By the use of an optical device that doubled and separated the images, Biessel's disclosure permitted the patient to compare, on a simultaneous and side-by-side basis, two images and to pick the clearest image. The Biessel disclosure taught that minimizing the separation of the images such that they remained within the central foveolar region of the retina, or about 60 milliradians of angular separation, provided the patient with greatest ability to detect differences in image quality.

Because Biessels taught the placement of spherical and cylindrical lenses of different power in the image paths, the device was limited to creating comparative images that differed only in spherical and cylindrical modulations of their wavefronts. Another deficiency of the U.S. Pat. No. 3,240,548 disclosure was that it, like the other prior art cited above, had to be placed in immediate proximity to the patient's eye, thereby potentially inducing instrument accommodation and inaccurate measurements. For these reasons the use of Biessel method was not appropriate for incorporation in the Applicant's '644 disclosure.

U.S. Pat. No. 7,963,654 to Aggarwala taught a method and apparatus for comparing two images on a side-by-side basis that incorporated two optical channels with identical objects that produced images whose wavefronts could be spherically modulated in an independent fashion by the use of a Badal optical slide. The disclosure taught a means for the patient to select the clearer of the two images and this selection was then used to adjust the optics in the device to create the next comparative side-by-side test. When a reversal occurred, the refractive measurement at a single meridian was recorded. By measuring two or more meridians of the eye, the subjective manifest refraction, limited to sphero-cylindrical terms, could be determined. Aggarwala's disclosure was limited to testing one meridian of the eye at a time, it offered no provision for modulating the wavefront of images with higher order aberrations beyond sphere and cylinder, it was placed in close proximity to the patient, and it required a computation of the predicted depth of field in order to determine the measurement resolution. Because of these deficiencies, the use of the image comparison method taught by Aggarwala is not appropriate for use in the Applicant's '644 invention.

It will be clear from the description that follows that the Applicants' disclosure provides novel inventive features and overcomes limitations and deficiencies of the prior art referenced above. The Applicants' disclosure provides the eye care professional with a new and improved method and apparatus for vision testing and for simulating the vision that will result from an eyesight correcting modality. The invention permits patients to compare, effectively simultaneously, images that would be formed by corrective products with different optical specifications.

It will also be evident from the following description that the Applicant's invention permits optical attributes of corrective modalities other than wavefront modulation to be effectively demonstrated, or simulated, to the patient on an effectively simultaneous and optional, side-by-side basis. These other optical attributes include the optical quality of the corrective lenses that result from the dispersive qualities of the lens material, known by those skilled in the art as the Abbey number. Other optical attributes that can be simulated and compared include the images produced by anti-reflective, photo-chromic and other premium spectacle lens coatings compared to images created by products that lack these attributes. The difference in images produced by lenses with a high index of refraction vs. lenses with a low index of refraction can also be simulated. Corrective lenses that have these and other optical attributes are now increasingly available, yet prior art methods of vision testing listed offer no means to demonstrate the benefits of these attributes or the quality of eyesight that they provide. These and other deficiencies are resolved with the Applicants' novel apparatus and methods as taught herein which allow patients to preview, compare, and select a specification for a vision correcting product that will best meet their individual needs.

### SUMMARY

A vision testing method is disclosed for generating a plurality of images to be viewed by a patient, modulating the wavefront of one or more images by an amount that differs from another image and/or changing optical attributes other than the wavefront of an image by an amount that differs from another image, and selecting the preferred image or images based upon patient response.

### BRIEF DESCRIPTION OF THE DRAWINGS

FIG. 1 is a diagrammatical side elevational view of the apparatus with patient seated in the exam chair

FIG. 2 is a perspective view of the patient chair and rear tower

FIG. 3 is a partial top plan view of the wavefront generators for the right and left eyes with the adjustable lenses removed

FIG. 4 is a partial detailed view of the wavefront generator for the right eye with the adjustable lenses in position

FIG. 5 is a table listing the identity of the adjustable lens elements shown in FIG. 4.

FIG. 6 is a block diagram of inputs and outputs of the system computer.

FIG. 7 is a diagrammatical side elevational view of the apparatus showing two wavefront generators for the right eye.

FIG. 8 is a perspective view of the patient's view of the viewport mirror and wavefront generators.

FIG. 9 is a perspective view of the patient's view of the viewport mirror, the image generators and wavefront generators that are active in producing images viewed by the patient's right eye.

FIG. 10 is a perspective view of the patient's view of the viewport mirror, the image generators and wavefront generators that are active in producing images viewed by the patient's left and right eyes under binocular viewing conditions.

FIG. 11 is a perspective view of the patient and near viewing apparatus.

FIG. 12 is the patient's view of the viewport mirror and the near viewing apparatus and images formed in them.

### DETAILED DESCRIPTION

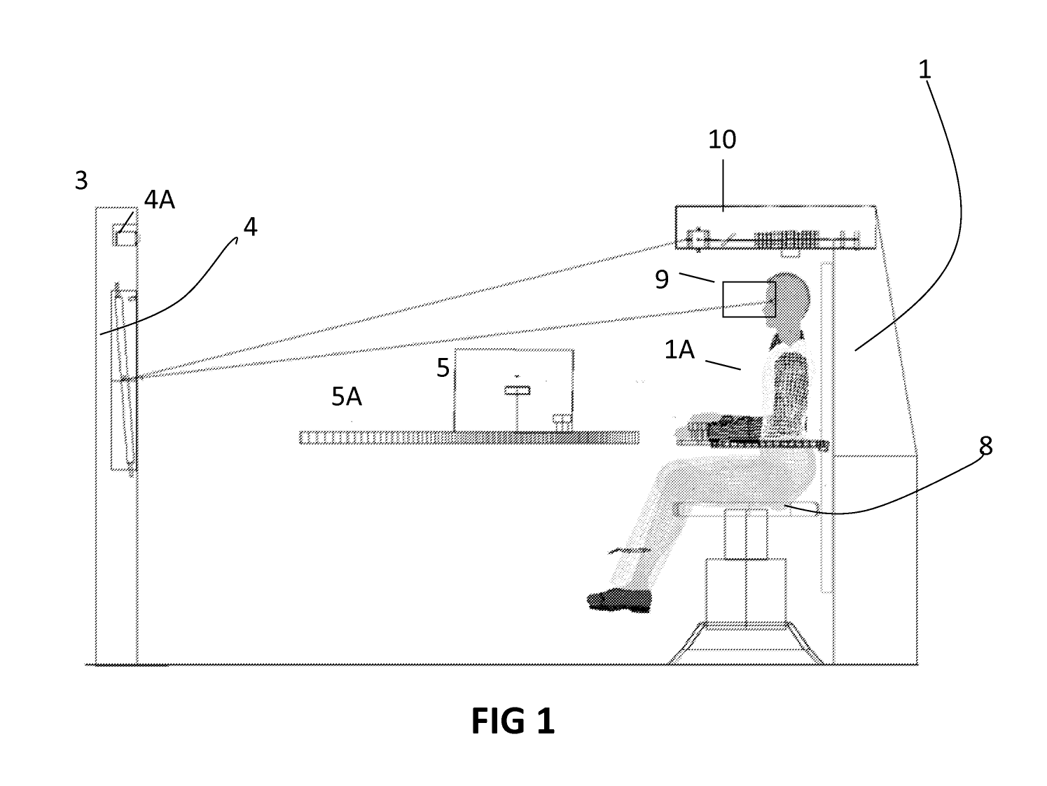

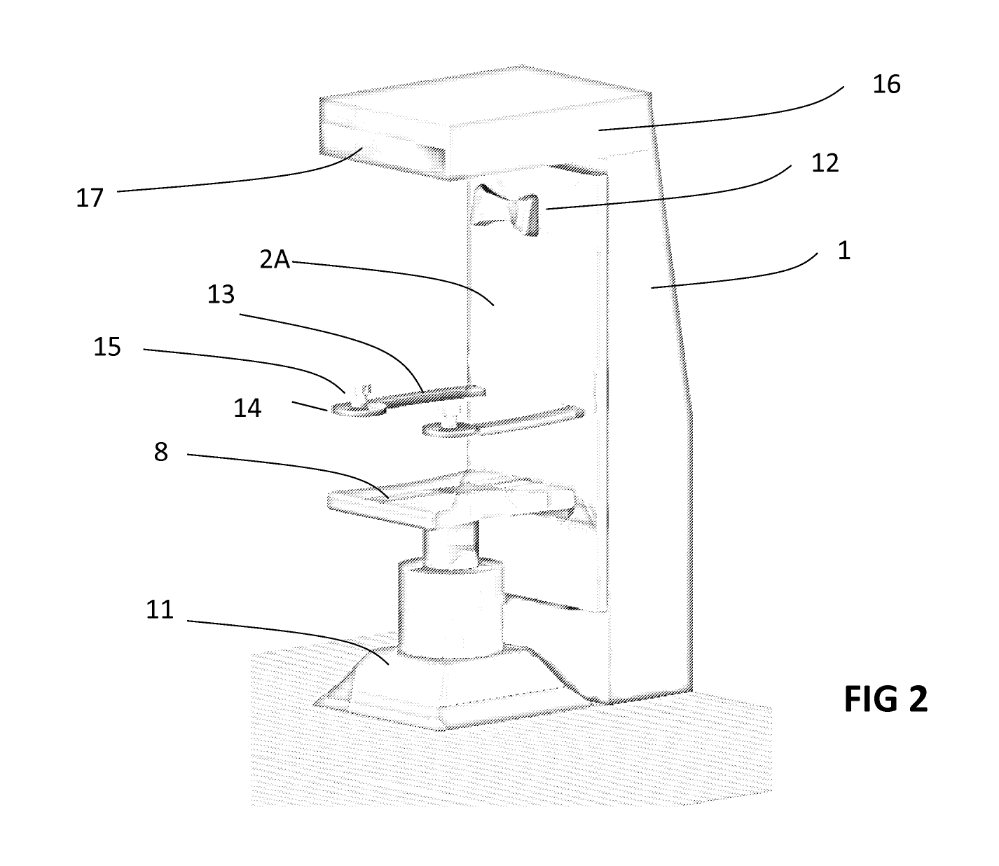

In general, the present apparatus is intended to be deployed in the examination lane of eye care professionals with typical, but non-limiting, dimensions of 8′×10.′ As shown in FIGS. 1 and 2, the apparatus consists of tower 1, an examination chair 2A, a viewport 3 which houses a reflective field mirror 4 and one or more optional cameras 4A, and an operator control terminal 5. The patient 1A undergoing vision testing with the apparatus is seated in the examination chair seat 8 which is adjusted to place the patient's eyes within the desired examination position noted by box 9. Images are generated by wavefront generators 10A or other means in the optical tray 10 and directed to a field mirror 4 in the viewport 3 where they are reflected to the patient's eyes located within the desired examination position 9. Behind the patient, rear cabinet 1 houses a computer, power supply, and other specialty electronics to control the wavefront generators, located in optical tray 10. Images projected from the optical tray are reflected by field mirror 4 and viewed by the patient.

FIG. 2 shows a perspective view of the examination chair 2A that is located adjacent, and forward of, the vertical tower 1, and it is preferentially mechanically isolated from the tower 1 so that patient movements in the chair are isolated from the optical components in the tower. The examination chair has a seat portion 8, the position of which is adjustable through motor means located in the base of the chair 11 that may be made responsive to the system computer. The seat back has a head rest 12 that may be adjustable through manual or by automatic means made responsive to the system computer. Optional head restraint (not shown) may be deployed from the underside of optical tray 10 to aid in stabilizing the patient during the exam.

The examination chair has arm rests 13, each of which has a platform 14 for supporting patient input means 15. In one preferred embodiment, the input means is a rotary haptic dial that the patient may rotate, translate, or depress to provide input to the system computer during the examination. Suitable haptic controllers are manufactured by Immersion Technologies, San Jose, Calif. 95131, and such controllers are particularly suited for patients to provide intuitive input to the system during the exam. Numerous other input devices are known, such as a mouse, a joystick, a rotary control, touch-sensitive screen, voice, and other control means, any of which may be employed as alternative embodiments for use with the present apparatus.

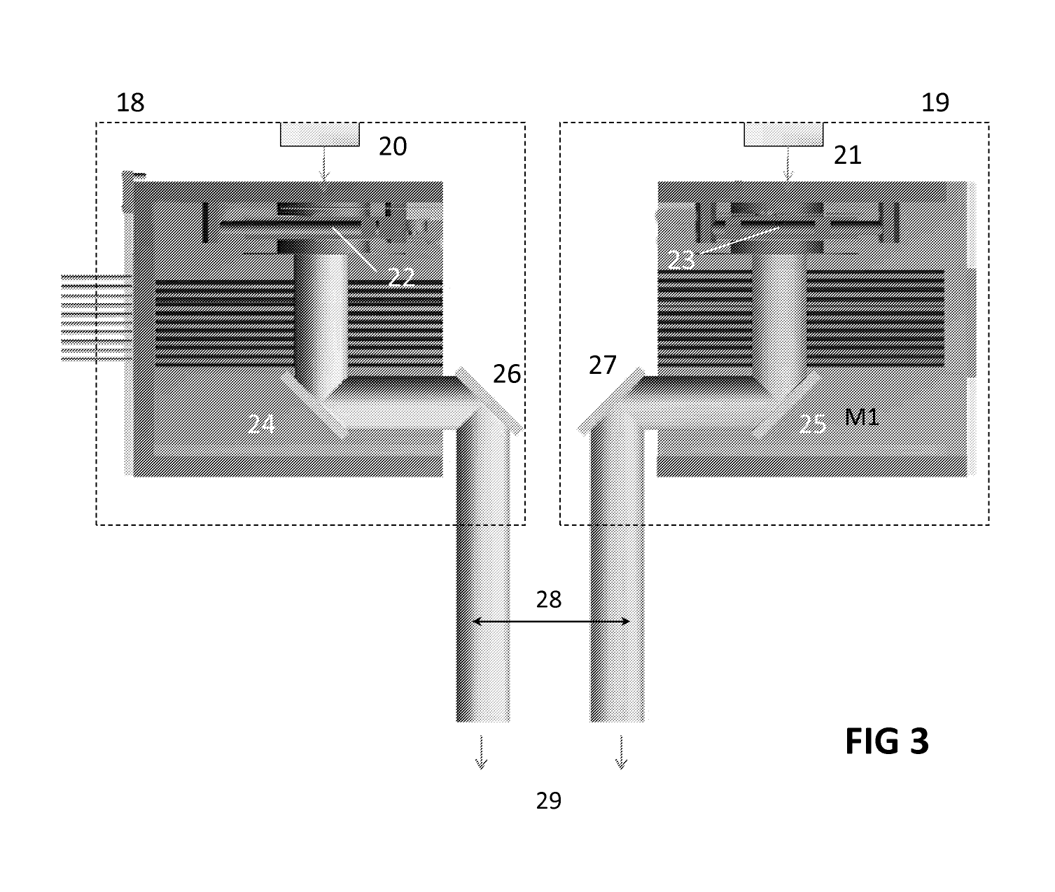

FIG. 3 shows a top view of the wavefront generators for the right eye 18 and left eye 19 with the adjustable lenses and accessory lenses removed. Display means for the right eye 20 and left eye 21 generate images. One suitable image generating means is model SXGA OLED-XL™, made by EMagin Company, Bellevue, Wash. Numerous other image generating means and modalities are known in the art including LED, OLED, DLP, CRT and other means, any and all of which may be suitable for alternative embodiments for use with the present apparatus.

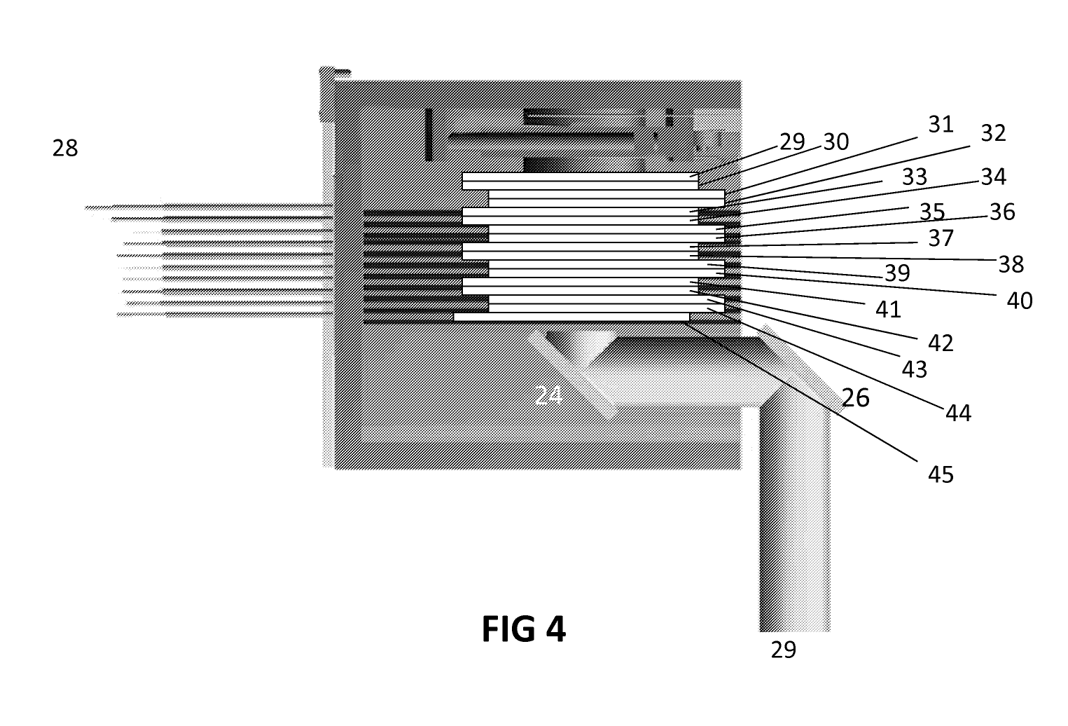

Images generated by 20 and 21 pass through collimating lenses 22 and 23. Collimated light of the images then traverses the stack of adjustable Alvarez lens elements and accessory lens elements, shown in detail in FIG. 4, and described below, where they are redirected by beam turning mirrors 24 and 26 for the right eye, and by beam turning mirrors 25 and 27 for the left eye where they are then directed towards the field mirror 29. The position and angle of lenses 24, 25, 26, and 27 are made responsive to the system computer in order to direct the beam to the field mirror and to adjust the spacing between the left and right beam paths to that of the patient's inter-pupillary distance, 28. Suitable adjustable lenses for the apparatus are lenses described by Alvarez in U.S. Pat. No. 3,305,294. These lenses consist of pairs of lens elements, each of which has a surface shape that can be described by a cubic polynomial and each element is a mirror image of its fellow element. As the lens elements are made to translate relative to each other in a direction that is perpendicular to the optical axis of the element, the optical power imparted to an image passing through them changes as a function of the amount of translation. The lenses are mounted in surrounding frames and they are translated by motion means (not shown) such that their movement is responsive to the system computer. The wavefront of the image is changed as it traverses each lens element. The total change imparted as the image exits the last optical element of the wavefront generator is referred to herein as the modulation of the wavefront of the image. Such modulation can also be effected by other suitable optical means known to those skilled in the art.

It is known to those skilled in the art that the co-efficients of the equations that define the shape of the Alvarez lens elements may be optimized to improve their optical performance, by, for example, using suitable optical design software such as ZeMax (Radiant ZEMAX LLC, 3001 112th Avenue NE, Suite 202, Bellevue, Wash. 98004-8017 USA). Such modifications of the adjustable lenses to improve their performance are fully envisioned within the scope of the present disclosure.

Other types of adjustable lenses and mirrors are known in the art that may be used in the wavefront generator to modulate the wavefront of the image and they are considered to be within the scope of the disclosure. Deformable mirrors that may be made responsive to a computer are known such as those manufactured by Edmunds Optics, 101 East Gloucester Pike, Barrington, N.J. 08007-1380. As one alternative embodiment, the adjustable Alvarez lenses described above may be replaced by fixed lenses, by one or more deformable mirrors, or by any combination of fixed lenses, deformable mirrors, and Alvarez lenses and remain under the scope of the disclosure. In another alternate embodiment, one or a plurality of discrete lenses, disposed in a rack or other arrangement, may be substituted in order to modulate the wavefront of the image.

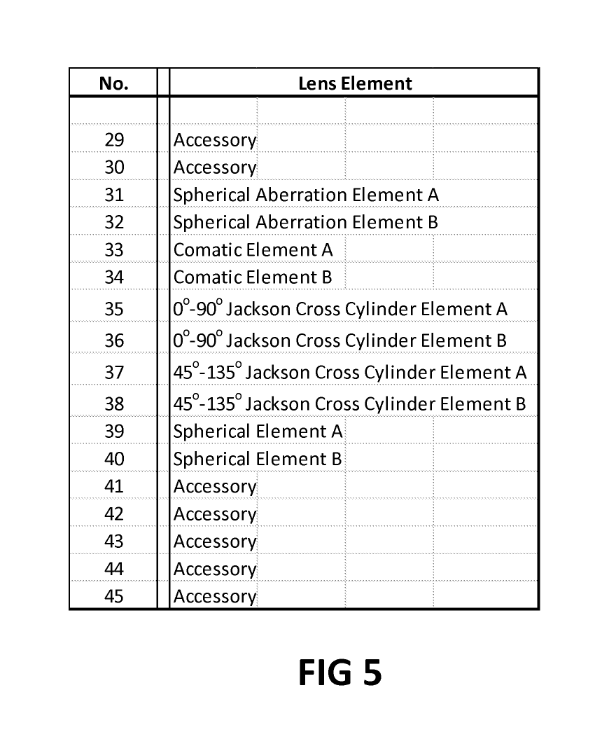

FIG. 4 shows a more detailed view of the wavefront generator for the right eye showing the adjustable Alvarez lens pairs and the accessory lens pairs 29-45 that are used to modify the wavefront of the image that is created by display means 20. The identity of these lenses is shown in FIG. 5.

In one preferred embodiment, the relationship between the linear separation of the Alvarez lens elements and the spherical modulation of the wavefront of the image has been found to be 2.1 mm=1 D, and for the linear separation of the Alvarez lens elements and the cylindrical modulation of the wavefront of the image has been found to be 1.8 mm=1 D.

A suitable magnetic or optical position encoder (such as provided by Renishaw's Encoder Read Head T 1 0 0 1 15 A and Encoder Scale A-9420-0006M) may be placed on the bottom of lens elements 29-45 and a signal sent to the system computer for use in determining the location of the lens elements. Such means may be employed for calibration or for continuous operation purposes.

In general, it is envisioned that the optical elements listed in FIG. 5 will be selected to modulate the wavefront of the image to provide a full range of modulation of the wavefront in sphero-cylindrical fashion from −20 D to °20 D and astigmatic corrections up to, or beyond, 8 D. The apparatus is also capable of providing continuously adjustable sphero-cylindrical wavefront modulations in any increment desired by the operator in ranges between 0.005 D to 20 D increments. This continuously adjustable wavefront modulation of variable resolution is a major improvement over the prior art HVA, the phoropter, and other prior art because high resolution steps (e.g. 0.01 D) can be selected to provide very fine wavefront modulations to achieve optimal vision and to create specifications for corrective eyewear at much higher resolution than conventional ophthalmological eyeglass prescriptions that are limited to 0.125 D and 0.25 D resolution. By providing this inventive feature, the present apparatus can provide specifications for corrective eyewear to a resolution that the new generation of spectacle lens fabrication technologies can now accurately create. Such variable resolution is also useful for the operator to set the apparatus to low resolution steps (e.g. 1.0 D) in certain situations such as examining patients with low vision in order to speed their vision exam.

In addition to modulating the spherical and cylindrical components of the wavefront of the image, the wavefront generator described herein is able to modulate the wavefront to achieve the correction of higher order aberrations such as spherical aberration by directing the motions of lens elements 31 and 32 and comatic aberrations by directing the motions of lens elements 33 and 34. As one alternative embodiment, the wavefront generator may utilize fixed and adjustable lens elements to modulate spherical and astigmatic errors and deformable mirror elements to modulate higher order aberrations of the wavefront of the image.

In addition to modulating the wavefront in a spherical, cylindrical, and higher order fashion, optical attributes of the image other than the wavefront of the image may be imparted through the use of accessory lens elements 41-45. For example, to emulate the effect of an image of a horizontally polarized filter added to a spectacle lens, a similar polarized filter may be introduced into one of the accessory lens channels 41-45. Similarly, to demonstrate the optical effect of anti-reflective lens coatings, an appropriate anti-reflective lens coating plate can be inserted into accessory lenses 41-45.

FIG. 1 shows a side view of the viewport 3, which houses the field mirror 4. In one preferred embodiment, the field mirror is round in shape and has a spherical concave curvature with a radius of curvature approximately 2.5 M and a diameter between 10″ and 24.″ Such mirrors are known in telescopic applications and a suitable mirror may be procured from Star Instruments, Newnan, Ga. 30263-7424. Alternative embodiments for spherical mirrors are known such as CFRP (carbon fiber reinforced polymer) spherical rectangular mirrors which may be procured from Composite Mirrors Applications in Arizona.

Alternative embodiments for the field mirror include the use of an aspheric mirror, a toroidal mirror, a mirror that is non-circular in shape, and a plano mirror.

In one preferred embodiment, the radius of curvature of the mirror corresponds to the approximate distance between the spectacle plane of the patient's eyes (at the optimal testing position 9) to the mirror, and from the center of the lenses in the wavefront generator to the field mirror. It is known to those skilled in the art that a real object placed at a distance that is twice the focal length (or at the radius of curvature) of a concave spherical mirror will produce a real inverted image of the object with a magnification of one, or “unity magnification.” In this configuration, the object and image are said to occupy conjugate planes, a property of lenses and mirrors that is well known to those skilled in the art. Stated differently, it can be said that when the object and its image occupy conjugate planes, the optical properties of the object in the object plane are reproduced exactly by the image in the image plane as if the physical object itself was located in the image plane. It can also be said that the object has been optically relayed to the conjugate image plane.

An inventive feature of the U.S. Pat. No. 3,874,774 patent was the recognition that the optical relay property of concave mirrors could be applied to corrective optical lenses as well as physical objects. Specifically, Humphrey recognized that the corrective power of the adjustable Alvarez lenses located at a distance equal to the radius of curvature of the concave field mirror would be effectively relayed to a position equidistant from the concave mirror at the conjugate image plane. When the patient's spectacle plane was located at the center of curvature of the field mirror and the corrective adjustable lenses were the same distance away (albeit at a slightly different angle relative to the mirror), then the properties of the corrective adjustable lenses would be optically relayed to the patient's spectacle plane.

It will also be apparent to those skilled in the art that operating the apparatus at, or near a condition of “unity magnification” (i.e. when the correcting lenses and the patient's spectacle lenses are located a distance from the concave spherical field mirror a distance that is equal to the radius of curvature) is one preferred embodiment. However, it is known that changes in effective lens power that result from the adjustable lenses imaged at non-unity magnifications may be compensated for by the following equation:

Po=Pc(M)²

where Po is the effective power of the lens at the patient's spectacle plane, Pc is the power of the corrective lenses in the wavefront generator, and M is the magnification, given by Do/Di, where Do is the distance between the corrective lenses and the field mirror and Di is the distance between the field mirror and the patient's eyes. This relationship may be employed to adjust Po when the patient's eyes are at distances from the field mirror other than a distance equal to the radius of curvature of the field mirror.

As shown in FIG. 1, a desk 5A is provided to support the display terminal 5 used by the operator to provide control inputs to the computer and to receive displays from the device. Operator input to the system may be provided by conventional keyboard, mouse, or optional haptic means 15 to control the apparatus during the examination. These devices are connected to the system computer through conventional cable, fiber optic, or wireless means. Other input means are known to those skilled in the art such as voice and gesture input and these and other inputs are considered to be within the scope of the disclosure.

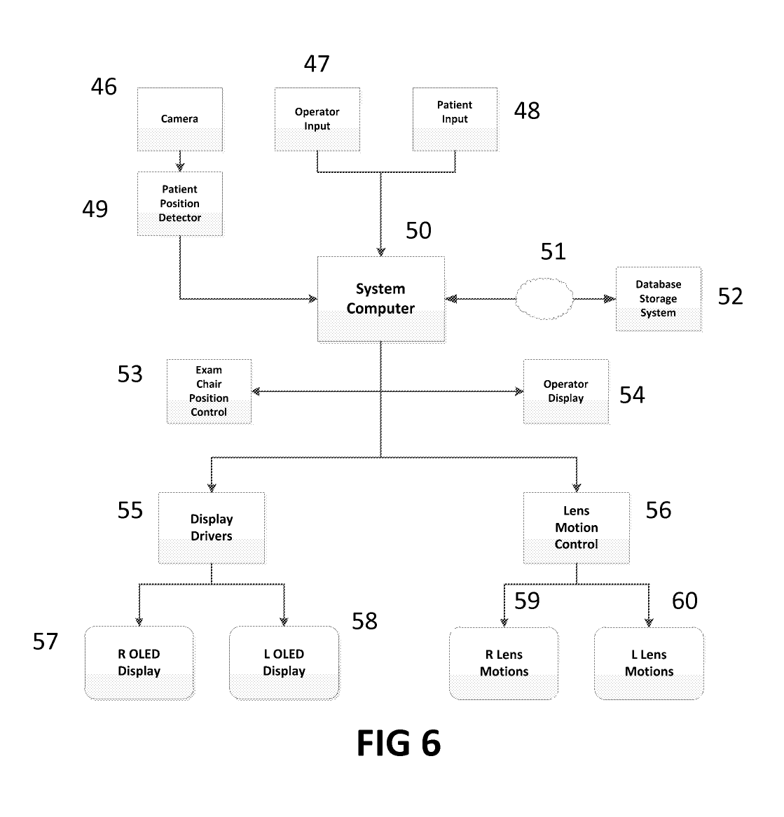

FIG. 6 shows inputs and outputs of the system computer 50 to different subsystems of the apparatus. Camera 46 provides information to the patient position detector 49, which provides input to system computer 50. Operator inputs 47 and patient inputs 48 are provided to the system computer.

The system computer 50 receives inputs and provides outputs to database storage system 52, which in one preferred embodiment may be transmitted through the Internet 51.

The system computer 50 provides outputs to display drivers 55 which run the digital displays 57 and 58 which, in one preferred embodiment, may be organic light emitting diodes described above. The system computer 50 provides outputs to lens motion control system 56 which directs the actuators that drive the adjustable lenses for the right and left channels of the wavefront generators, 59 and 60, respectively.

In one preferred embodiment, information from one or more cameras 4A can be sent to an appropriate eye tracking software such as (SmartEye created by Smart Eye AB in Gothenburg, Sweden; Tobbi created by Tobii Technology AB in Danderyd, SWEDEN; or faceLAB from Seeing Machines Inc in Tucson, Ariz.) to determine the distance between the patient's eyes and the viewport mirror. Once this distance is known, the formula listed above can be used to calculate the effective power of the lens at the patient's actual position. Such a feature allows the patient to move freely within a defined range 9 while the system automatically calculates the correction to be applied to the effective power of the lenses in the wavefront generator. This is one significant inventive feature over the prior art, as it allows the patient to test under natural viewing conditions and be free to move about without the need to be restrained by a forehead or head rest. It also improves the accuracy of the measurements by ensuring that the proper calibration factor is applied based upon the actual position of the patient.

This formula can provide corrective conversions through calibration tables and/or by adjusting the lenses in the Alvarez stack 25A to correct for the operation of the device at such non-unity magnifications. Such corrections may be made by the system computer automatically without input by the operator. It is also known that only one location in the Alvarez stack can be at the center of curvature, and that correction factors must be applied to the lenses in the stack that are located adjacent the center of curvature. To further enhance the calibration and accuracy of the apparatus, a wavefront sensor, such as a spatially resolved refractometer, or Hartmann Schack device, may be placed in the locales that can potentially be occupied by the patient's eyes during testing. By placing the wavefront sensor in each locale in box 9 and by setting the wavefront generator to produce its full range of wavefront modulation at each locale, it is possible to provide calibration or correction values for each locale and degree of wavefront modulation.

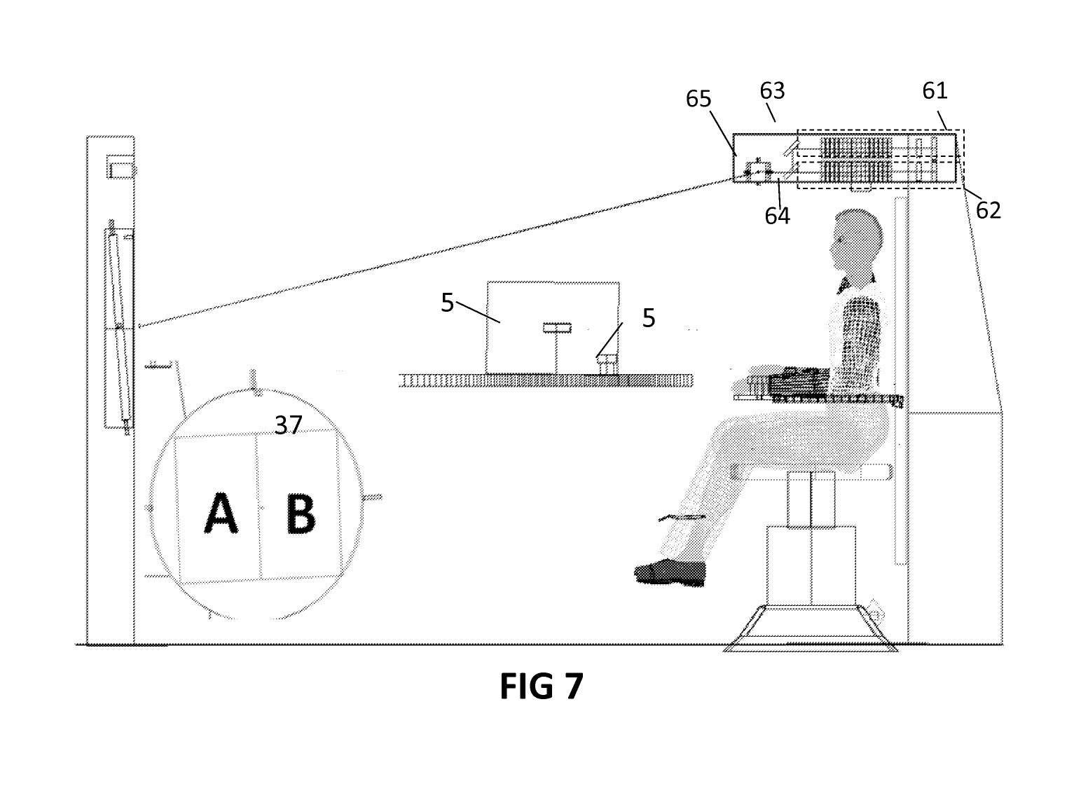

Referring to FIG. 7, it is seen that a preferred embodiment features wavefront generators 61 and 62 that are directed to field mirror 4 to form images A and B in 37 in the right eye of the patient. In a preferred embodiment, the images generated by 61 and 62 are substantially identical as they pass through wavefront generators 61 and 62. If 61 and 62 impart different wavefront modulations to the image, then the patient will view these images in the viewport as having distinctions if the patient's visual system can detect differences in appearance of the images. Stated differently, the patient may perceive that image A looks different than image B, or that images A and B are indistinguishable.

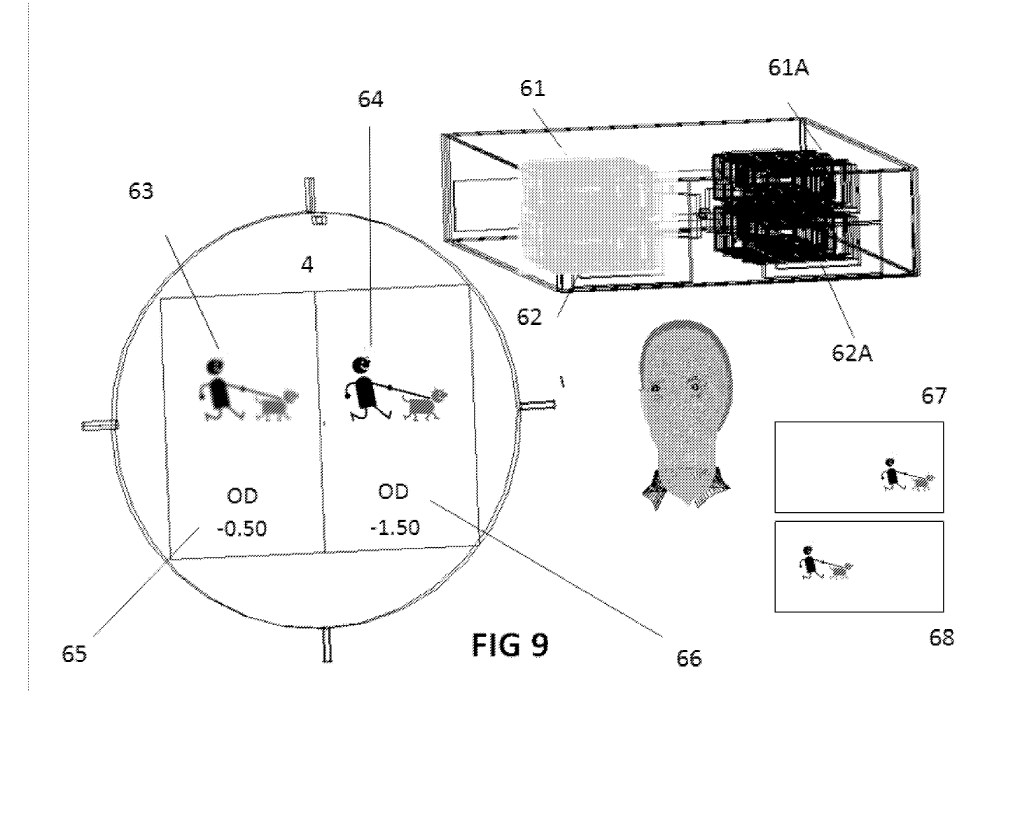

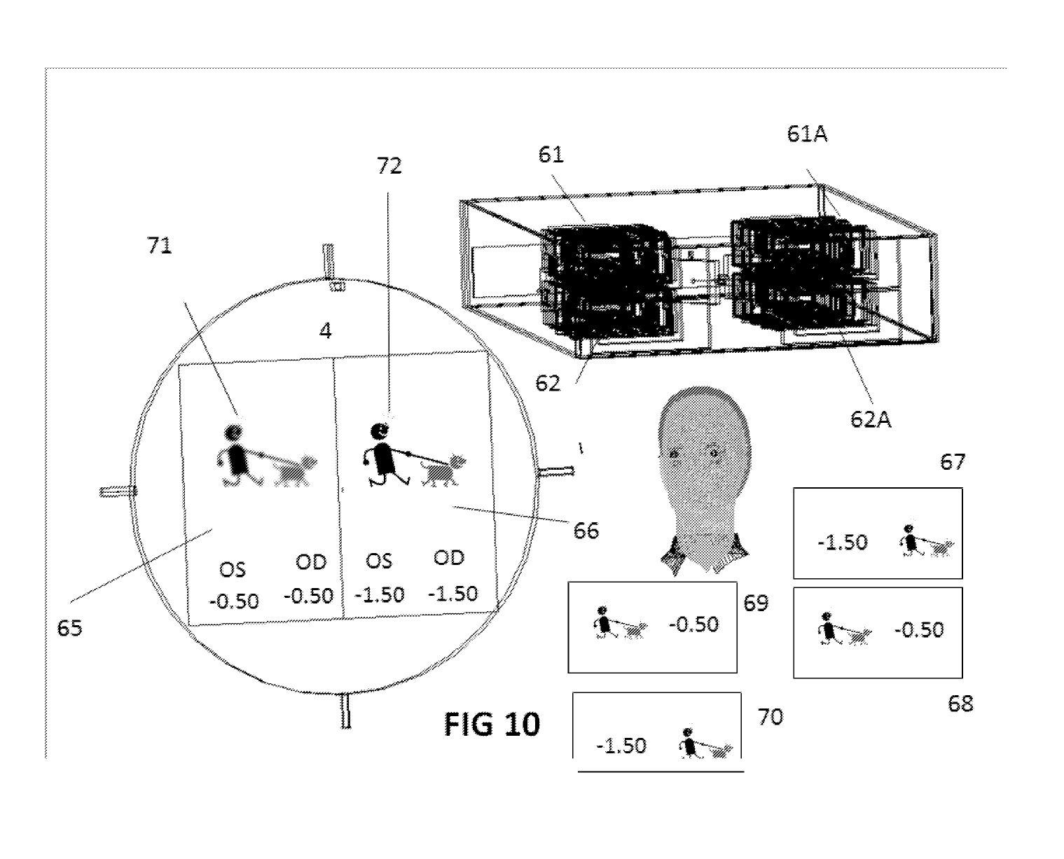

FIG. 9 shows how an identical image, that of a man walking his dog, can be created identically, by image generators 67 and 68, but then the images are subjected to different wavefront modulations by wavefront generators 61A and 62A with spherical modulations of −0.50 D and −1.50 D, respectively. When these wavefront modulations, imparted by 61A and 62A respectively are relayed to the spectacle plane of the eye by the relay mirror 4, it appears to the patient as if he is viewing the image through two different optical corrections that are presented on a side-by-side and simultaneous basis. Because of this presentation, the patient can quickly and easily determine which of the two presented images, 63 or 64 is the clearest and preferred. The system provides input means 48 for the patient to designate his preference. Whilst FIG. 9 showed the selection under monocular conditions, FIG. 10 shows a similar selection made by the patient under binocular viewing conditions in which wavefront generators 61 and 62 create images for the left eye and wavefront generators 61A and 62A create images for the right eye. It is fully intended for the device disclosed herein to operate in either monocular or binocular viewing conditions for substantially simultaneous comparison of images.

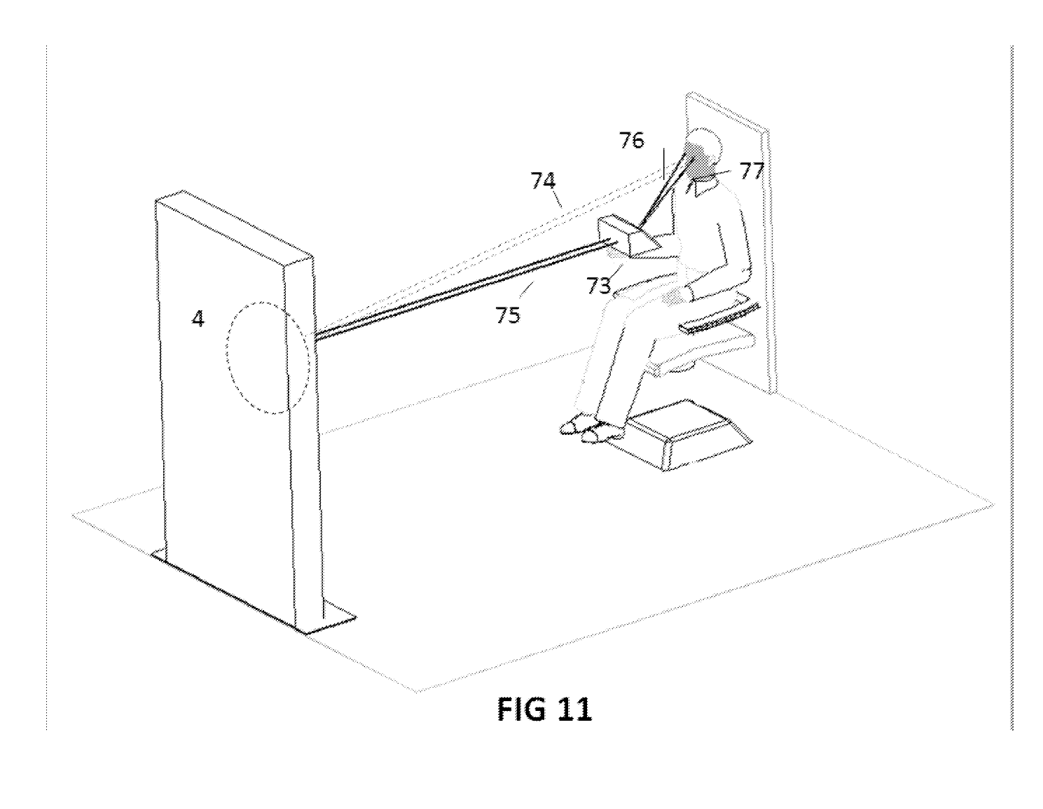

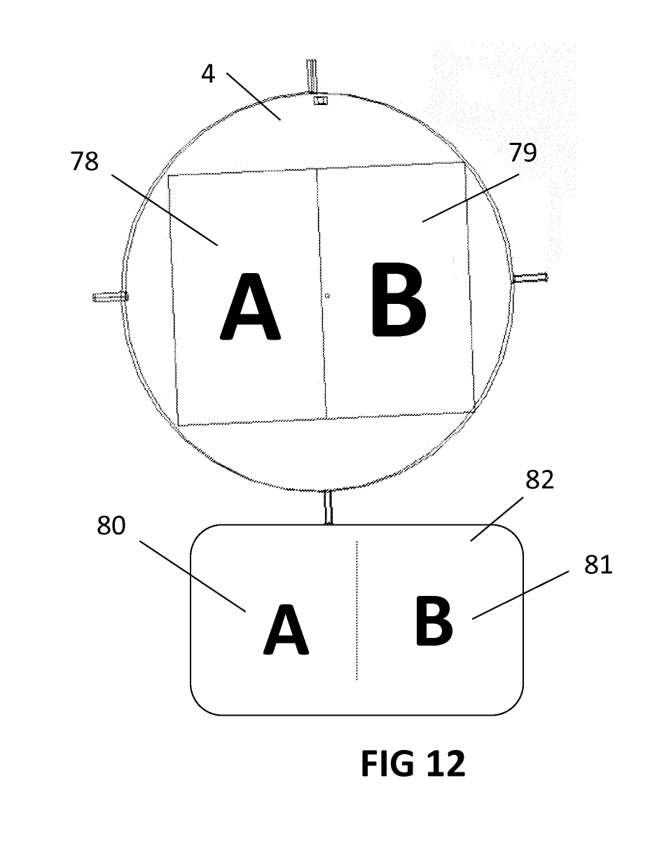

In FIG. 11, the use of the invention with a near viewing 73 accessory is shown. This accessory has diverting mirrors (not shown) that cause the images to diverge such that they appear to emanate from the partially transparent plane of the viewing plate 82. The effect of this presentation is seen in FIG. 12 that shows the patient's view of the distance (viewport) 4 and near (near viewing accessory) images in 82. This allows the patient to preview, compare, and select prescription A and prescription B in a simultaneous basis at both far and near distances.

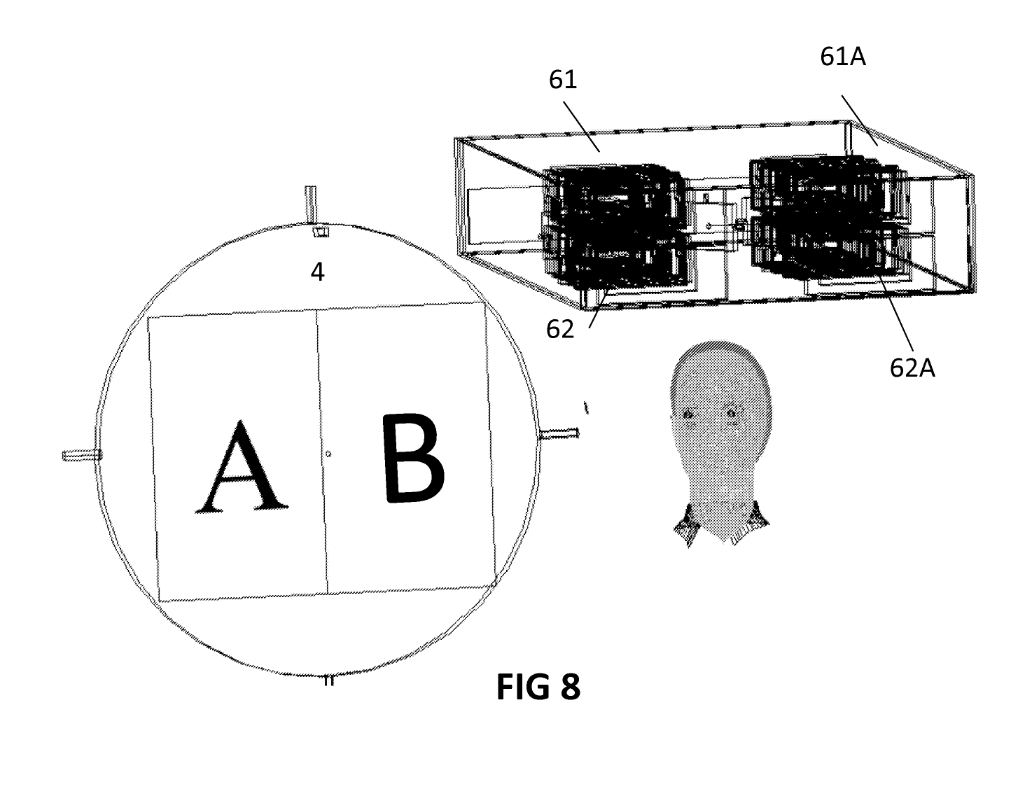

FIGS. 7-10 describe an embodiment of the device that employs two separate image and wavefront generating means for producing two images for evaluation by the patient. Alternative embodiments of the device may feature one image generation means which is subsequently split into two images by a suitable beam splitter known in the art and then subjected to wavefront modulation by an appropriate optical system. An alternative embodiment of the device incorporates a single image generating and single wavefront generating channel in which a single image is generated and then subjected to different wavefront modulations by rapidly moving the lenses in the wavefront generator. In this manner, the image is subjected to different wavefront modulations on a temporal rather than spatial basis. Yet another embodiment of the device would subject a single image to temporally separated wavefront modulations as described above, in addition to spatial separation of the image by a suitable optical scanner or similar means. The persistence of vision is known to those skilled in the art and rapidly scanned images may be employed in order for the patient to compare images on a substantially side by side basis, although they are actually created on the retina in separate time intervals. Embodiments that incorporate such time-based-multiplexing using the flicker fusion threshold of the subject as the basis for selecting the time interval to display the images in a substantially simultaneous fashion to the patient are within the scope of the invention.

Thus, it can be seen that the present device provides a means for a patient to preview, compare, and select between one or more real-time images while the system computer compiles the results for each selected image. The data obtained is used by the doctor to prescribe a corrective lens or lenses, or to provide the information necessary for corrective surgical procedures such as LASIK.

While methods and apparatuses for vision testing, and modifications thereof, haven been shown and described in detail herein, various additional changes and modifications may be made without departing from the scope of the present disclosure.

We claim:

1. A vision testing method in which the patient to be tested is in a natural viewing position with nothing interposed between the patient's eyes and the image being viewed comprising the steps of:
generating a plurality of images to be viewed by a patient for comparison, modulating the wavefront of one or more images by an amount that differs from another image, and comparing and selecting the preferred image or images based upon patient response.

2. A vision testing method as defined in claim 1 comprising the additional step of selecting a specification for a corrective modality based on said preferred image.

3. A vision testing method as defined in claim 1 comprising the additional step of computing a laser vision treatment profile based upon the wavefront modulation of the said preferred image.

4. A vision testing method as defined in claim 1 in which at least two of said images are generated in a side-by-side arrangement for comparison by the patient in a monocular or binocular fashion.

5. A vision testing method as defined in claim 1 in which said images are modulated by at least one wavefront generator.

6. A vision testing method as defined in claim 5 in which said wavefront generator has at least one lens element.

7. A vision testing method as defined in claim 5 in which said wavefront generator has a plurality of lens elements.

8. A vision testing method comprising the steps of: generating one or more images to be viewed
by a patient, changing optical attributes other than the wavefront of the image of one or more of the
images by an amount that differs from another image, and selecting the preferred image or images
based upon patient response.

9. A vision testing method as defined in claim 8 comprising the additional step of selecting a specification for a a corrective modality based on said preferred image.

10. A vision testing method as defined in claim 8 in which at least two of said images are generated in a side-by-side arrangement for comparison by the patient.

11. An apparatus for vision testing comprising devices for generating a plurality of images to be viewed by a patient, said images being projected to the retina of the patient so as to appear substantially simultaneously devices for modulating the wavefront of one or more images by an amount that differs from that of another image, and devices for selecting the preferred image or images based upon patient response.

12. A vision testing apparatus as defined in claim 11 in which at least two of said images are generated in a side-by-side arrangement for comparison by the patient.

13. A vision testing apparatus as defined in claim 11 in which said images are modulated by at least one wavefront generator.

14. A vision testing apparatus as defined in claim 13 in which said wavefront generator has at least one lens element.

15. A vision testing method as defined in claim 13 in which said wavefront generator has a plurality of lens elements.

16. An vision testing apparatus comprising devices for generating one or more images to be viewed by a patient, devices for changing optical attributes other than the wavefront of the image of one or more images by an amount that differs from another image, and devices for selecting the preferred image or images based upon patient response.

17. A vision testing apparatus as defined in claim 16 in which said images are modulated by at least one wavefront generator.

18. A vision testing apparatus as defined in claim 17 in which said wavefront generator has at least one lens element.

19. A vision testing apparatus as defined in claim 17 in which said wavefront generator has a plurality of lens elements.
